(19) United States
(12) Patent Application Publication
(10) Pub. No.: US 2019/0314591 A1
(43) Pub. Date: Oct. 17, 2019

(54) SECRETION CLEARING ENDOTRACHEAL TUBE (SCET)

(71) Applicant: John Allen Pacey, Vancouver, WA (US)

(72) Inventor: John Allen Pacey, Vancouver, WA (US)

(21) Appl. No.: 16/387,447

(22) Filed: Apr. 17, 2019

Related U.S. Application Data
(60) Provisional application No. 62/659,055, filed on Apr. 17, 2018.

(51) Int. Cl. A61M 16/04

(52) U.S. Cl. CPC A61M 16/0463; A61M 16/042; A61M 2205/32; A61M 16/0486; A61M 16/0445

(57) ABSTRACT

A ventilation catheter and airway management system is described to create conditions that tend to clear secretions during the normal conduct of ventilation by having an exclusive outflow channel and/or to use a programed cuff leak combined with PEEP to protect the airway. The secretion clearing endotracheal tube may include separate inflow and outflow paths as part of a double lumen, to facilitate breathing when placed in a patient. The double lumen portion intersecting to form a single lumen for protrusion into the intratracheal region allowing the new inspiratory fluid from the airway management system to flow into a patient and the used inspiratory fluid from the patient to flow back to the airway management system. A pharyngeal suction component removes fluid from the intratracheal region via periodic suctioning.

### PRIORITY CLAIM

This application claims priority to Provisional Application No. 62/659,055 filed Apr. 17, 2018; the contents of this application are hereby incorporated by reference as if fully set forth herein.

### RELATED APPLICATIONS

The following patent applications are hereby incorporated by reference in their entireties as if fully set forth herein: U.S. application Ser. No. 14/284,799 filed May 22, 2014, titled “SECRETION CLEARING VENTILATION CATHETER AND AIRWAY MANAGEMENT SYSTEM”; U.S. application Ser. No. 16/351,392 filed Mar. 12, 2019, titled “SECRETION CLEARING VENTILATION CATHETER AND AIRWAY MANAGEMENT SYSTEM”; and “U.S. Pat. No. 9,839,755 filed Jan. 27, 2014, titled “SECRETION CLEARING PATIENT AIRWAY MANAGEMENT SYSTEM.”

### COPYRIGHT NOTICE

This disclosure is protected under United States and/or International Copyright Laws.© 2018, 2019 Dr. John Allen Pacey. All Rights Reserved. A portion of the disclosure of this patent document contains material that is subject to copyright protection. The copyright owner has no objection to the facsimile reproduction by anyone of the patent document or the patent disclosure, as it appears in the Patent and/or Trademark Office patent file or records, but otherwise reserves all copyright rights whatsoever.

### FIELD OF DISCLOSURE

The current disclosure is directed to secretion clearing endotracheal tubes and related devices and techniques.

### BACKGROUND

The evolution of a new belief that secretion clearing capability can, and should, be built into Ventilating systems is supported by the notion that having repeated passages of suction apparatus into the airway can only increase the risk of transmission of bacteria into the trachea and lungs. The principle causes of Ventilator Acquired Pneumonia are the commensal bacteria that exist in the patient normally but also those common in ICU and Hospital environs (such as MRSA), the accumulation of airway secretions which act as a growth medium, and the periodic suctioning necessary to clear such secretions.

Secretion clearing strategies have been attempted in the past such as the suction system of Fry above the ETT cuff and later the Mallincrodt Evac® endotracheal tube which features a subglottic suction port that has been placed just above the endotracheal tube cuff. This has been tested pre-clinically by ventilation of a Porcine subject and was shown to be effective by removal of pooled secretions residing above the cuff. The current belief is that the periodic escape of fluid past the cuff downward into the trachea is a contributing cause of contamination of the airway. Early studies demonstrated a modest benefit in reducing VAP using these suction devices above the cuff but this is questioned in some current studies.

The Evac tube was the first specialized endotracheal tube that has been recognized to reduce VAP. This strategy of suctioning just above the cuff proved less effective than other interventions and is replaced by the tapered cuff and other strategies as by Perez in U.S. Pat. No. 9,907,920.

### BRIEF DESCRIPTION OF THE DRAWINGS

FIG. 1 illustrates an example secretion clearing endotracheal tube having various features.

FIG. 2-4 illustrates different views of a secretion clearing endotracheal tube having a cuff crease.

FIG. 5 illustrates another example secretion clearing endotracheal tube having a metal layer on at least a portion of the endotracheal tube.

### DESCRIPTION OF THE PREFERRED EMBODIMENT

In some aspects, the purpose of the described devices and related methods is to create conditions that tend to clear secretions during the normal conduct of ventilation by having an exclusive outflow channel and/or to use a programed cuff leak combined with PEEP to protect the airway. One significant benefit of described devices and methods is the relative simplicity of production which has benefits providing low cost of goods for manufacture. The system is largely automated in that it uses forces usual for the airway such as PEEP for its successful operation. It is noteworthy that great expense and great research efforts have been expended to upgrade and perfect the Ventilator systems but virtually nothing has been expended to improve the final connection of the Ventilating system to the patient. Therefore for 100 years or more we have been extending the use of single lumen endotracheal tubes without questioning whether improvements can or should be made.

This application is intended to describe one or more embodiments of the present disclosure. It is to be understood that the use of absolute terms, such as “must,” “will,” and the like, as well as specific quantities, is to be construed as being applicable to one or more of such embodiments, but not necessarily to all such embodiments. As such, embodiments of the disclosure may omit, or include a modification of, one or more features or functionalities described in the context of such absolute terms. In addition, the headings in this application are for reference purposes only and shall not in any way affect the meaning or interpretation of the present disclosure.

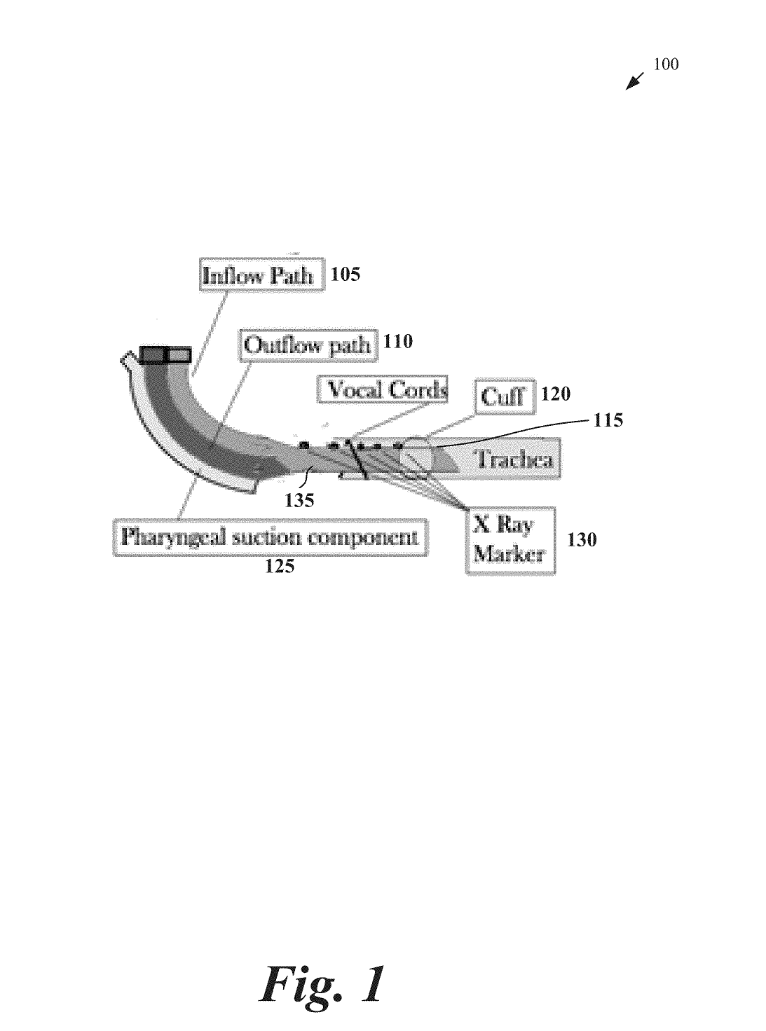

FIG. 1 illustrates a Secretion Clearing Endotracheal tube (SCET) or device 100. SCET 100 may include an inflow path 105 and outflow path 110, to facilitate breathing when placed in a patient. A first end 115 of the inflow path or tube 105 may be placed into the trachea of the patient proximate to the patient's vocal cords. SCET 100 may also include a cuff portion 120 that is located at or near the first end 115 of the inflow and outflow paths 105, 110. The cuff portion 120 may be preferably placed into the trachea of the patient.

In some variants, the SCET may include a pharyngeal suction component 125 that is positioned at least partially along the inflow path 105, the outflow path 110, or a combination thereof. The pharyngeal suction component 125 may optionally perform pharyngeal suction, which may improve visibility and scavenge hazardous smoke produced from the surgery, for example. In some aspects, the pharyngeal suction component may be activated periodically or based on need to suction Saliva and Esophageal reflux secretions to keep the Pharynx free of contaminants and to collect that which is expelled by PEEP air flow passing through the controlled leak from the cuff.

In some examples, the pharyngeal suction component 125 may be made of PVC and may have a 3 mm to 5 mm internal diameter. This internal diameter may be beneficial because some secretions are viscous and may come about in considerable volume. Blood may also contaminate the airway with associated clotting residues. Blood may especially be a product of micro laryngeal or laser surgery. In some aspects, the pharyngeal suction component and may operate by application of external suction as required. The pharyngeal position of the suction may prevent fluids from gathering following reflux, and intercept fluids that may be expelled by a “Programmed Cuff Leak” described herein. The “Programmed Cuff Leak” is controllable, for example, by variations of PEEP, Cuff Pressure, Monitoring leak % on the ventilator, and variations of FIO2.

In some aspects, the SCET 100 may also include one or more x-ray markers 130. In many cases, the accurate placement of the Secretion clearing tube (SCET) 100 may be important because the cuff must lay in the tracheal lumen without resting on the laryngeal cords and the area of the bifurcation likewise best not be in contact with the cords for an extended period of time. The ETT 135 of the SCET 100 may have a number of Radiographic or x-ray markers 130 at 1 cm intervals to locate cuff position with respect to the vocal cords. In other examples, various numbers of markers 135 may be used, for example, depending on different spacing of the markers 135, total length of the SCET 100, and for various other design reasons. The marker spacing may, in some cases, be more or less than 1 cm, such as a single market designed to target the Vocal cords exactly. Other spacing may be selected for pediatrics or special applications. The markers 130 may enable accurate placement of the SCET 100 upon x-ray examination of the chest and neck of the patient. An alternative method of confirming the position may be performed using Video Laryngoscopy, for example, when Video Laryngoscopy is used to initially place the endotracheal tube (ETT) 135.

In some cases, multiple radiographic markers may be implemented that identify the location of the Bifurcation and also the cuff itself such that the vocal cords are positioned midway from the Cuff to the Bifurcation.

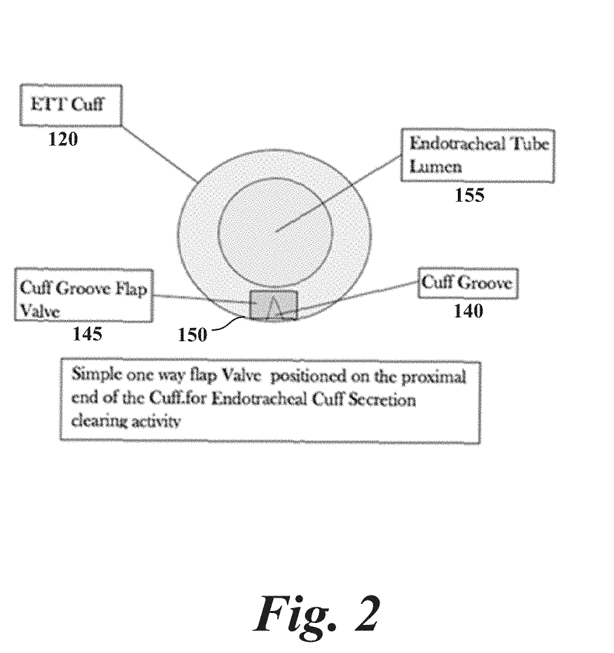

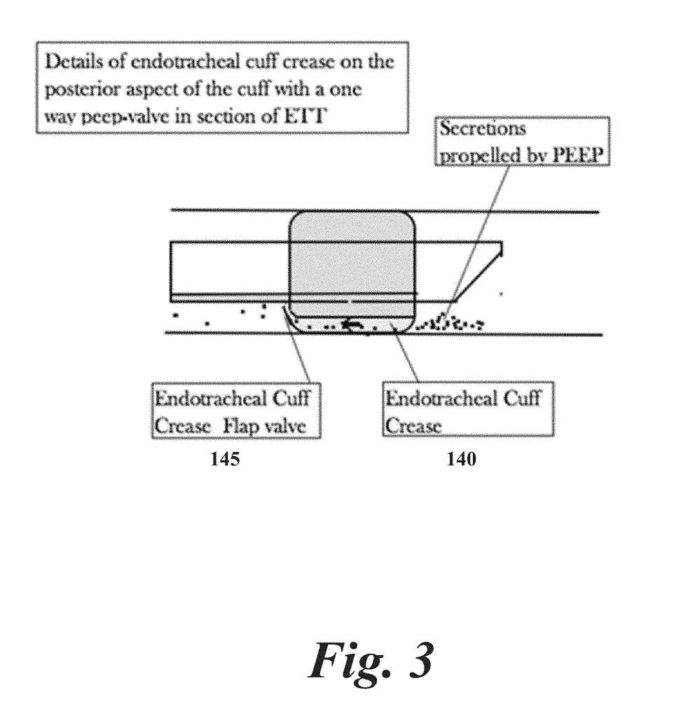

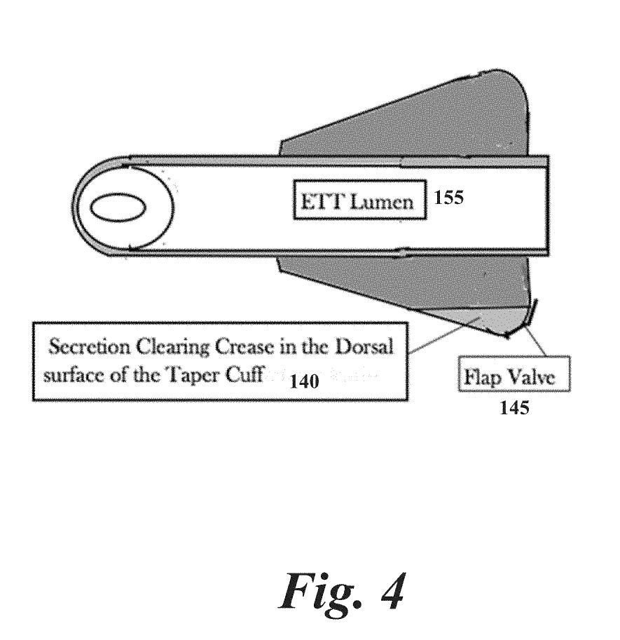

As further illustrated in FIGS. 2-4, in some aspects, the SCET 100 may include a programmed cuff leak option. This innovation operates with a small cuff leak measured by the ventilator and propelled by PEEP (continuous end expiratory positive airway pressure). In this aspect, a small cleft, crease, or groove 140 in the cuff 120 serves to propel any tracheal secretions past the ETT cuff 120 and toward the pharynx. The placement of a small flap valve 145 on the external end 150 of the cleft or groove 140 and on opposite the endotracheal tube lumen 155 impedes flow from the pharynx into the trachea, which is advantageous because there is substantial proof that more effective tapered cuff designs have superior control. This extra flap valve protection is created to assure that flow is not permitted toward the trachea via the small cleft in the dorsal part of the endotracheal cuff 120.

In some examples, the cuff flap 145 may be made of PVC, and may be 1 to 2 mcm in size or length. The cuff flap 145 may have a durometer that assures protection. In other cases, the cuff flap 145 may be constructed as a ball valve type of occlusion secured to the cuff. The main security will be provided by modest PEEP.

In some aspects, the cuff crease feature positioned on the posterior aspect of the cuff may allow some limited leakage of the Gas delivered by the ventilator so that secretions may bypass the cuff and pass into the pharynx. In some cases, the cuff crease may be designed so that the amount of leakage permitted is generally 10% or less of the Total inspired volume so that a slow continuous liberation of secretions may pass along with CO2 and air.

In some aspects, the cuff crease may include a one-way way flap or ball valve that permits fluid to exit but not pass back below the cuff. In some cases, the cuff crease may be powered or driven or moved by clinical PEEP or constant positive airway pressure.

In some examples, the cuff crease may be controlled to allow 0-10% of inspired volume to pass by means of increasing or decreasing cuff pressure. In some cases, the cuff crease may be designed for the Tapered Endotracheal tube Cuff series.

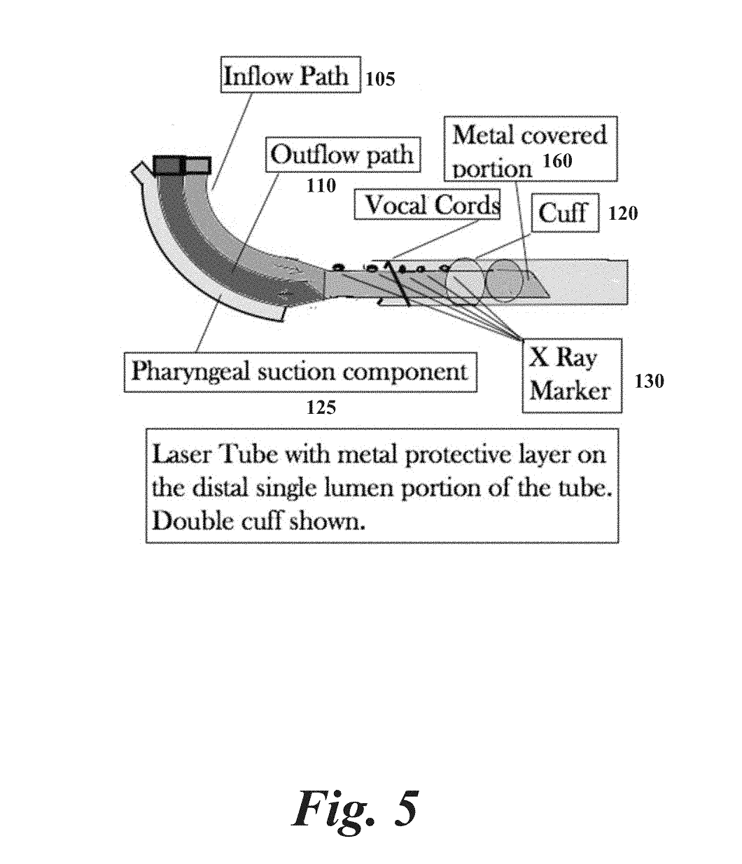

FIG. 5 illustrates another example secretion clearing endotracheal tube having a metal layer 160 on at least a portion of the endotracheal tube. In some cases, the SCET design may be used as a Metal armoured Laser Surgery device. Laser surgery may benefit from the smaller size of the distal single lumen component of the SCET which passes through the vocal cord area. This distal part of an SCET type of tube may in fact be the only part of the tube that may be made from metal components. SCET may also be equipped with the typical double cuff safety design that is recognized for fire prevention ability. The efficiency of the SCET may also result in superior ventilation performance. The SCET may be equipped with the optional pharyngeal suction which would be an advantage to improve visibility and scavenge hazardous smoke and blood produced from the surgery. In some examples, the Secretion clearing tube may be configured with a distal trans laryngeal portion that is protected by metal for laser operating conditions and may have a double cuff safety feature for fire prevention.

In some aspects, the ETT may be coated or covered or made of conventional wrapping metals. In some aspects, the metal layer be up to 1.5 mm thick. In some aspects, only a portion of the ETT may be covered or made of metal, such as the portion of single lumen design that will be transiting through the vocal cord area, or approximately the distal 50%.

There are a multitude of additional benefits that may be provided by one or more features of the described Secretion Clearing tube over the use of traditional mono lumen tubes. Some, but by no means all, of the beneficial uses of the described device is provided below.

In some aspects, the SCET may be used as a “T-Piece” replacement. For example, the SCET may be used as a “Classic T-Piece” for weaning patients from Mechanical Ventilation (MV), for example, with the advantage that there is minimal dead space and the flow of enriched oxygenated air passes down to a lower level of the ventilation catheter while the patient breaths from his/her own volition. This is done by at once passively introducing humidified oxygen enriched air via the inflow limb of the (SCET) and allowing exhausted air to pass out via the exhaust limb. This is usually done with a classical mono-lumen ETT and the “T Piece” is an external cap that is placed over the open end of the ETT when the tube is disconnected from a ventilator. The process allows caregivers to evaluate the strength of patient breathing activity while medication is withheld. The SCET has the advantage of promoting mixing of inflow and outflow gas to make the transition to non-mechanically assisted breathing more likely to succeed while also aiding the patient to clear secretions that might otherwise accumulate in the mono-lumen tube during such a trial due to the continuous passage of ventilating air and oxygen.

In some cases, the SCET may be equipped with the optional pharyngeal suction which would be an advantage to improve pharyngeal secretions management.

In some examples, the SCET may also be used for PSV during weaning from ventilator care. The PSV may provide benefits over the “T Piece” implementations. PSV is an alternative to the use of a “T-Piece” strategy wherein a mechanical assist for the weaning may be more effective in preserving patient strength.

In some aspects, the SCET may be used as an aid to Microlaryngeal surgery. For example, a surgeon doing micro-laryngeal surgery requires ventilation that allows an un-hurried operating environment but also benefits from the use of a small caliber tube passing through the operative field. The SCET will permit a smaller size tube to be used because the inflow/outflow gasses will pass more readily through the shorter dead space zone and will have a more enriched oxygen to deliver. The size can be expected to go down by 1-2 mm, for example, from 6 to 5 or 4 mm. The Secretion clearing tube may have a selection of small diameter distal single lumen segments that have small 4, 5, 6 mm, diameters that are designed for microlaryngeal surgical procedures. This may greatly add to the convenience of the surgeon. This allows some use also for upper tracheal surgery where stenosis or other abnormality make a smaller tube diameter an advantage. In some cases, use of some PEEP coupled with a modest cuff leak may keep the airway more completely oxygenated and may clear CO2 more completely.

In some cases, the SCET may be used as an armoured ETT. For example, the use of the SCET to replace classic armoured tubes may be effective. The upper double lumen portion of the SCET is stronger and very resistant to kinking during patient positional change. The position of the tube with respect to the cords should be known at all times.

In some cases, the secretion clearing tube may be protected from endotracheal tube obstruction by the presence of a controlled cuff leak which acts as a relief valve for any blockage of the exhalation limb of the secretion clearing tube. This may prevent barotrauma to the lungs. In yet some instances, the secretion clearing tube which may be used for ventilation with sub-standard FiO2 in the range of 15-20% and may be used in a way that will produce a modest High CO2 environment (40-50 mmhg.) that will be less likely to support combustion.

Cleaning strategies: there are cleaning devices available that are very effectively designed to clear secretions that may be too viscous or bloody to be cleared by normal means. This may be applied to the exhaust limb of the tube. Such a device is the Endo Clear device.

In some aspects, it is possible to run an anesthetic safely with a low oxygen high CO2 environment in or with the described SCET. For example, by running the anesthetic in the SCET (e.g., the inflow path, the outflow path, or the pharyngeal suction component), low oxygen levels, such as between 10-20%, which is the typical percentage in ambient air, may be present to provide a safe environment. This may substantially reduce the risk of an airway fire when using a laser for surgery, for example.

In some aspects, the SCET may utilize and include a flexible ski tip to assist with endotracheal tube passage (as described in U.S. Pat. No. 5,873,362 to Parker, which is hereby incorporated by reference as if fully set forth herein). This may provide the added benefit of hosting a micro atomizer feature incorporated in the tip for airway cleaning. In some cases, an airway pressure sensor may be included on or proximate to the flexible ski tip to permit periodic adjustment of cuff pressures and ventilator performance

In some aspects, the SCET may include or incorporate a fluidic passage for saline or periodic use of perflurocarbon and/or a separate suction channel.

The embodiments of the disclosure in which an exclusive property or privilege is claimed are defined as follows:

### REFERENCES

The following references are also incorporated by reference in their entireties as if fully set forth herein:
U.S. Pat. No. 9,907,920 B2 to Li et al., filed Nov. 30, 2015 and issued Mar. 6, 2018, titled “ENDOTRACHEAL TUBE WITH DEDICATED EVACUATION PORT”;
U.S. Pat. No. 7,107,991 to Kolobow, filed March 2009, titled “ENDOTRACHEAL TUBE USING LEAK HOLE TO LOWER DEAD SPACE”;
US 2012/0097168 to Perez, filed April 2011, titled “ENDOTRACHEAL TUBE WITH SUBGLOTTIC SECRETION SUCTION AND DETACHABLE SUCTION PORT”;
U.S. Pat. No. 5,520,175 to Fry, filed May 1996, titled “ENDOTRACHEAL TUBE WITH SUCTIONING MEANS”
Endo Clear—Respir. Care 2014; 59(9):e122-e126 “Cochrane Review Pressure Support Versus T-Tube For Weaning From Mechanical Ventilation In Adults,” Magdaline T Ladeira, Flávia M R Vital, Régis B Andriolo, Brenda N G Andriolo, Álvaro N Atallah, Maria S Peccin, published 27 May 2014, by: Cochrane Anaesthesia, Critical and Emergency Care Group, DOI: 10.1002/14651858.CD006056.pub2
D'Haese J, DE Keukeleire T, Remory I, Van Rompaey K, Umbrain V, Poelaert J. “Assessment Of Intraoperative Microaspiration: Does A Modified Cuff Shape Improve Sealing?” Acta Anaesthesiol Scand, 2013; 57(7):873-880.

I claim:

1. A ventilation catheter configured to engage with the pre-pharyngeal/pharynx and intratracheal regions of an airway and for operably securing to an airway management system, the ventilation catheter comprising:
an open distal end for allowing new inspiratory fluid from the airway management system to flow into a patient and used inspiratory fluid from the patient to flow back to the airway;
a double lumen portion defining a first lumen pneumatically connected to provide an inspiratory fluid pathway for new inspiratory fluid and a second lumen pneumatically connected to provide an expiratory fluid pathway for used inspiratory fluid, the first lumen substantially parallel to the second lumen and configured to occupy the prepharyngeal/pharynx region;
a single lumen portion formed from an intersection of the first lumen with the second lumen for protrusion into the intratreacheal region, the single lumen portion including an open distal end for allowing the new inspiratory fluid from the airway management system to flow into a patient and the used inspiratory fluid from the patient to flow back to the airway management system; and
a pharyngeal suction component substantially parallel to at least one of the first lumen and the second lumen and configured to remove fluid from the intratreacheal region via periodic suctioning.

2. The ventilation catheter of claim 1, wherein the fluid comprises at least one of saliva and esophageal reflux secretions.

3. The ventilation catheter of claim 1, further comprising a cuff positioned about the single lumen portion, wherein the cuff comprises a cuff crease positioned about a posterior aspect of the cuff, wherein the cuff crease is configured to allow material to move from the trachea toward the pharynx.

4. The ventilation catheter of claim 3, wherein the material comprises tracheal secretions.

5. The ventilation catheter of claim 1, further comprising at least one radiographic marker positioned along the single lumen portion.

6. The ventilation catheter of claim 1, further comprising at least two radiographic markers positioned along the single lumen portion, wherein the at least two radiographic markers are spaced approximately one centimeter apart.
